(19) United States
(12) Patent Application Publication
(10) Pub. No.: US 2019/0380857 A1
(43) Pub. Date: Dec. 19, 2019

(54) ACTIVE HAND ORTHOSIS

(71) Applicant: Phoenix Innovation Kft., Pecs (HU)

(72) Inventor: Laszlo GELANYI, Budapest (HU)

(21) Appl. No.: 16/480,380

(22) PCT Filed: Jan. 10, 2018
(86) PCT No.: PCT/HU2018/050002

§ 371 Date: Jul. 24, 2019

(30) Foreign Application Priority Data
Jan. 25, 2017 (HU) P1700038

(51) Int. Cl. A61F 5/01

(52) U.S. Cl. CPC A61F 5/0118; A61F 2005/0155

(57) ABSTRACT

Active hand orthosis (20) for people with nerve-related paresis/paralysis of the hand, and for people with spastic paresis/paralysis of the hand, which comprises supporting elements (30) and moving elements (40) connecting the supporting elements (30), the moving elements (40) containing shape memory material, further comprising a control system (22), electric circuit elements (50) and a power source (60) for creating an activation signal inducing transformation of the shape memory material, and thereby deformation of the moving elements (40), characterised by that the moving elements (40) comprise: a wrist joint moving element (44), at least one finger moving element (46), and a thumb moving element (48), furthermore, in a state fitted to a lower arm (10) the supporting elements comprise: a lower arm splint (32) supporting a lower side of the lower arm (10), and leaving a wrist joint (11) free, a palm support (34) connected to the lower arm splint (32) by the wrist joint moving element (44), palm support (34) being provided at the proximal end of the fingers (15) opposable to the thumb (14), at least one finger support (36) connected to the palm support (34) by the at least one finger moving element (46), at least one finger support (36) being provided at the middle bone of the fingers (15) opposable to the thumb (14), and a thumb support (38) connected to the lower arm splint (32) by the thumb moving element (48), the thumb support (38) extending from the proximal end of the thumb (14) on the palm (13) to the lower bone of the thumb (14).

The object of the invention relates to an active hand orthosis for people with nerve-related paresis/paralysis of the hand, and for people with spastic paresis/paralysis of the hand.

An orthosis is a material, instrument or device that protects, fixes specific points of the body or provides support for them or lessens the burden on them. Such devices may be required in the case of peripheral nervous system diseases or musculoskeletal disorders, after an accident, or during or after performing active sports. An active orthosis is a device that does not only passively protect, fix, support, but also actively facilitates the movement of the affected area.

The object of the invention relates to an active orthosis that is able to substitute the movement of the wrist and fingers, and thereby facilitates the grasp-release function of the hand in the cases of persons who from birth, as a result of an accident or due to a stroke have peripheral or central nervous system or other nerve damage and are unable to use their hands and/or fingers, or only to a limited extent. In particular, the object of the invention relates to an anti-spastic hand orthosis that facilitates the movement of the fingers and wrists of persons with spastic paresis/paralysis of the hand as a result of flexor hypertonia. The term “fingers” may include the thumb as well, fingers other than the thumb are referred to as fingers opposable to the thumb.

### STATE OF THE ART

As a result of muscle tone increase (spastic hypertonia) the wrists and fingers of persons with spastic paresis/paralysis of the hand take on a convulsed, bent, deformed position, and patients are typically unable to bring the hand to a functional grasping position, due to which the affected hand is unable to grasp or touch.

Exoskeletal passive hand orthoses are commercially available that make use of mechanical tensioning elements to pull back the fingers and wrist into an unbent state. This results in the patient being able to grasp objects by intentionally tensioning the flexor muscles against the mechanical tensioning elements. Such a solution is provided by, for example, the hand orthosis distributed under the name SaeboFlex. The disadvantages of the solutions from the market are that these devices do not represent any assistance for patients who are completely unable to voluntarily flex their muscles.

This latter problem is overcome with the use of orthoses that have an exoskeletal robot system, which are connected to the fingers from the back of the hand and actively move them. The robotic orthoses that are commercially available are robust, cannot be worn in day-to-day life, and consume a great deal of energy. Most solutions, for example the “Powered Hand Exoskeleton” developed by the Berlin University of Technology (http://pdv.cs.tu-berlin.de/HandExoskeleton/HandExolntro.html), only facilitate movement of the finger bones, and do not ensure that important areas used during touching, feeling (such as the fingertips) are left free, nor do they ensure that the wrist returns to its functional grasping position. A further disadvantage is that the patient is unable to put on these glove-like devices without assistance.

A solution to the robust construction is represented by the orthotic glove described in patent number U.S. Pat. No. 9,387,112, in the case of which the movement of the joints is implemented with the use of a shape memory alloy (SMA). The shape memory alloy runs over the joints on the dorsal side of the hand (back of the hand) and connects rings placed on the fingers and a bracelet encompassing the hand. Although the size of the orthosis is significantly reduced by doing away with the robotic tensioning elements, the solution continues to have numerous disadvantages. The patient is unable to put on this orthotic glove without assistance either, and so the orthosis is not capable of supporting self-sufficient living.

Publication document number WO 2010/083389 A1 also discloses a hand orthosis, in the case of which the fingers are moved from above, i.e. from the back of the hand.

The inventor of the present invention recognised that the pulling/tensioning of the fingers and the wrist from above (from the back of the hand) has a damaging effect in case of spastically paralysed/weakened muscles. In the case of flaccid paralysis it is advantageous to move the fingers and wrist of hands from above, as in this way the palm remains free, however, in the case of spastic paralysis or paresis (weakening) due to the correction of the inherent convulsive position, the joints are subjected to a continuous pulling load, which even in the short term is damaging for the joints. Due to this effect the known orthoses are only recommended in the case of flaccid paralysis in which the cause of the paralysis is the pathological reduction of muscle tone, as in this case the pulling/tension exerted by the orthosis is weaker and is not of a constant nature, as in the flexor muscles there is no involuntary convulsive muscle contraction that has to be counteracted. It was recognised by the inventor of the present invention that in the case of a muscular spasm the metacarpophalengeal (MCP) joint must be supported, which has not been taken into consideration by the solutions according to the state of the art.

It was also recognised by the inventor of the present invention that fixing the fingers inside rings was also disadvantageous. The nerves run along the inner and outer sides of the fingers, which may be constricted by the ring pulled from above. The pulling of the ring also limits the microcirculation of the finger, which is also damaging.

The objective of the invention is to provide an active hand orthosis for people with nerve-related paresis/paralysis of the hand, and for people with spastic paresis/paralysis of the hand, which is free of the disadvantages of the solutions according to the state of the art.

It was recognised by the inventor of the present invention that the above disadvantages may be overcome if the fingers are not placed inside rings and are not pulled/tensed from above, but supported and lifted from the palm side. The cushioning of the fingers on the palm side ensures that the supports do not constrict the nerves or prevent microcirculation.

On the basis of the above recognition the objective was achieved by providing an active hand orthosis according to claim 1 and by providing a method according to claim 18.

Preferred embodiments of the invention are presented in the dependent claims.

Further details of the invention will be explained by way of exemplary embodiments with reference to figures, wherein

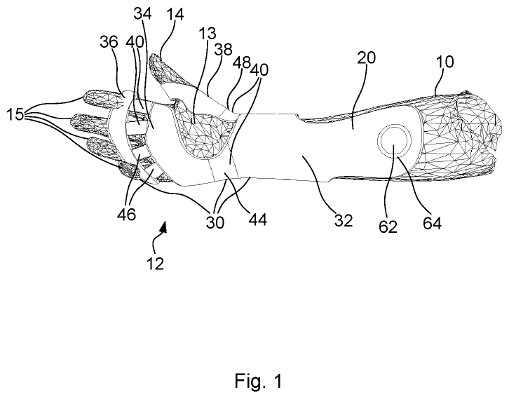

FIG. 1 is a schematic bottom view of a first embodiment of an active hand orthosis according to the invention as fitted to a lower arm,

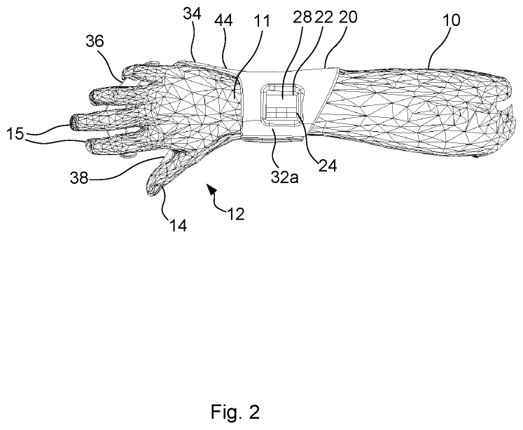

FIG. 2 is a schematic top view of the orthosis according to FIG. 1 as fitted to the lower arm,

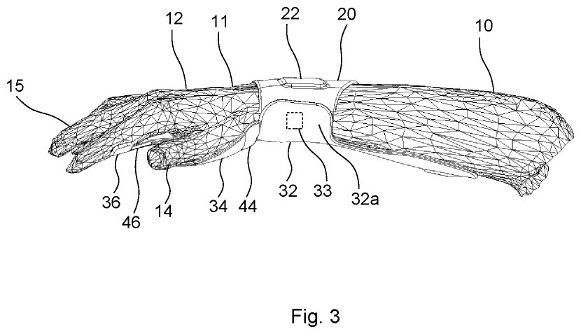

FIG. 3 is a schematic side view of the orthosis according to FIG. 1 as fitted to the lower arm,

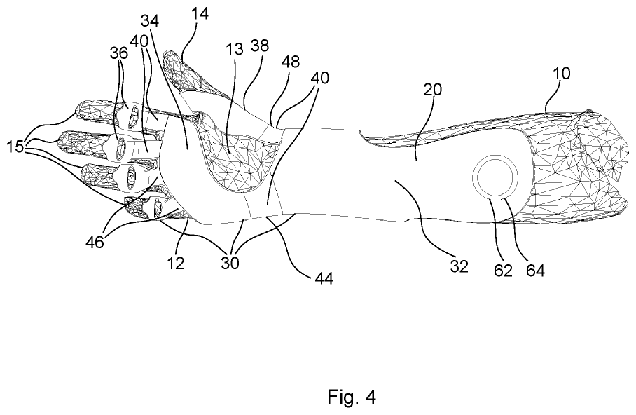

FIG. 4 is a schematic bottom view of a second embodiment of an active hand orthosis according to the invention as fitted to the lower arm,

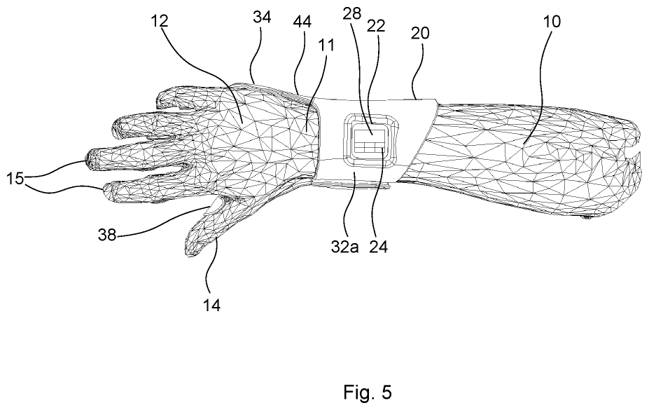

FIG. 5 is a schematic top view of the orthosis according to FIG. 4 as fitted to the lower arm,

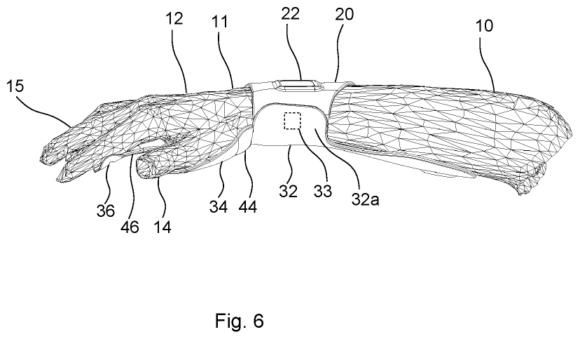

FIG. 6 is a schematic side view of the orthosis according to FIG. 4 as fitted to the lower arm,

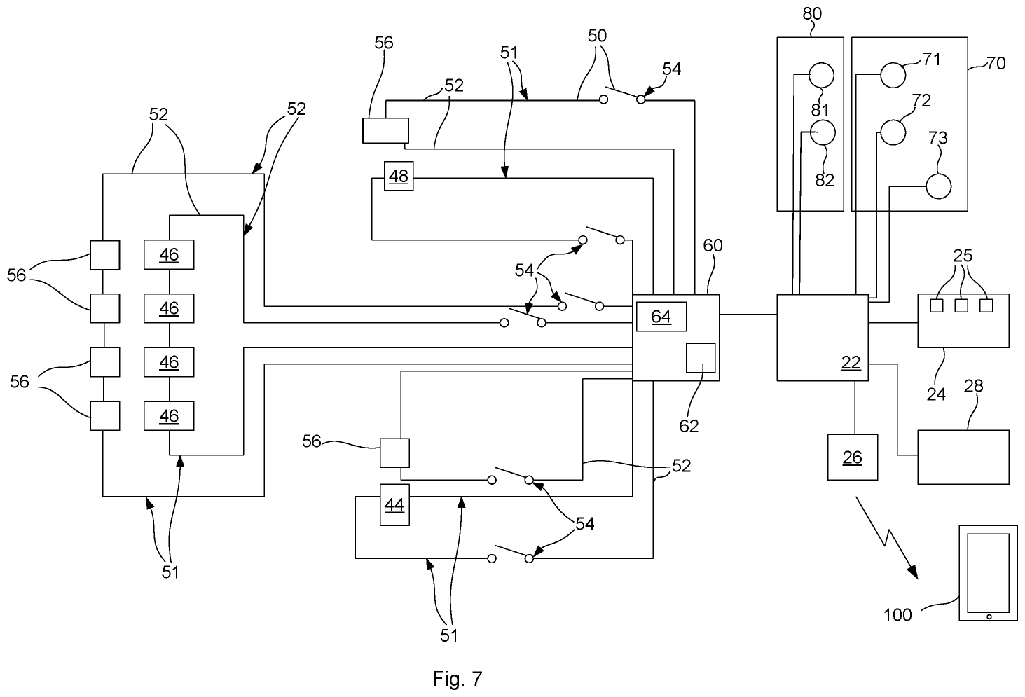

FIG. 7 is a schematic block diagram of the electric system of an active hand orthosis according to the invention.

FIGS. 1 to 3 illustrate schematic views of the first embodiment of an active hand orthosis 20 according to the invention. In the interest of better understanding the orthosis 20 is presented as fitted to the lower arm 10 of the arm of a user. The orthosis 20 has supporting elements 30 abutting the user's lower arm 10, the palm 13 of the user's hand 12 connected to the lower arm 10 through a wrist joint 11, the thumb 14 and the fingers 15 opposable to the thumb 14. The supporting elements 30 are connected to each other by moving elements 40 containing shape memory material.

In the case of the present embodiment the moving elements 40 contain a wrist joint moving element 44, four finger moving elements 46, and a thumb moving element 48.

The supporting elements 30 comprise:
a lower arm splint 32 supporting the lower side (palm 13 side) of the lower arm 10, leaving the wrist joint 11 free,
a palm support 34 connected to the lower arm splint 32 via the wrist joint moving element 44 at the proximal end, i.e. at the metacarpophalengeal (MCP) joint, of the fingers 15 opposable to the thumb 14,
a provided for the four fingers 15, which common finger support 36 is connected to the palm support 34 via the finger moving elements 46 and is arranged at the middle bone of the fingers 15 opposable to the thumb 14, i.e. between the proximal interphalangeal (PIP) joint and the distal interphalangeal joint (DIP), and
a thumb support 38 connected to the lower arm splint 32 via the thumb moving element 48, extending from the proximal end of the thumb 14 on the palm 13 to the lower bone of the thumb 14.

The lower arm splint 32 preferably has a fold-out strap part 32a that embraces the lower arm 10, and a locking system is preferably provided on the strap part 32a that secures the lower arm splint 32 in its position embracing the lower arm 10, preferably a magnetic lock. The latter is preferably a magnet 33 integrated in the two ends of the strap part 32a overlapping one another when in locked position, which are arranged so that they attract one another when in overlapping position. Naturally, other types of locking system are conceivable, such as press studs or a hook and loop fastener.

In the case of a common finger support 36 fewer finger moving elements 46 are sufficient, even just one, however, the orthosis 20 preferably contains at least two finger moving elements 46, one at each of the two outer fingers 15.

Such an embodiment is also conceivable in the case of which each finger 15 has a separate finger support 36 and a separate finger moving element 46, as illustrated in FIGS. 4 to 6.

In the case of the presented embodiment the thumb moving element 48 is positioned along the saddle joint, and moves the thumb 14 via this joint. However, such an embodiment is also conceivable in which the thumb support 38 may contain an additional moving element 40 (not shown) for moving the MCP joint at the thumb 14, in such a case the thumb support 38 consists of two parts, which are connected by this additional moving element 40. The above wording is understood to include this possibility as well.

Similarly, further supporting elements 30 may be arranged at the proximal finger bones of the fingers 15, in this case the finger moving elements 46 consist of a separate MCP joint moving part and a PIP joint moving part, which are connected together by this further supporting element 30. The above wording is understood to include this possibility as well.

Similarly, the DIP joints may also be separately moved using further support elements 30 and moving elements 40.

The orthosis 20 also contains a control system 22 serving for controlling the moving elements 40 containing the shape memory material. An activation signal initiating the transformation of the shape memory material serves to achieve the deformation (change of the shape) of the moving elements 40, and thereby to achieve the movement of the supporting elements 30 connected to the moving elements 40 with respect to each other. Such an activation signal may be a temperature signal, which may be produced by heating, or optionally cooling, the shape memory materials, or an electric signal, which may be produced with the creation of an electric field. Other types of activation signal are also conceivable, such as light (photon), magnetic signal, or some kind of chemical signal.

The shape memory material is a metal alloy, ceramic, polymer or polymer gel (or possibly other material) that regains its temporarily changed shape at a given temperature (at the so-called transit temperature), or due to the effect of an electric signal or other environmental signal.

In case of a shape memory material that can be activated with a temperature signal, this means that when the shape memory material is cooled below its transit temperature it becomes deformable again. In the case of a spastic hand it is the tensioning that has to be ensured because bending is created by the increased muscle tone on the bending side even in spite of the intent of the user. Therefore, it is preferable to configure the shape memory material such that as a result of the heat signal it takes the user's finger joints and wrist joint into the functional position (open position), from where due to the termination of the heat signal, optionally as a result of passive and/or active cooling, the spastic contraction of the muscles bends back the joints, and thereby deforms the shape memory material back to its initial position (grasping position), and as a result, the user grasps the desired object.

A further possibility is the use of bidirectional shape memory material. In the case of a bidirectional shape memory effect the shape memory material remembers two different shapes: a first shape belonging to a lower temperature, and a second shape belonging to a higher temperature. When cooled to the lower temperature such a material takes on the shape associated with it, while upon being heated to the higher temperature it takes on the shape associated with that temperature, in other words merely by changing the temperature (without any mechanical effect) its shape is changed between two states. The use of bidirectional shape memory material makes it possible to use the orthosis 20 even in the case of a hand/lower arm with flaccid paralysis, the natural muscle tone of which would not be capable of re-deforming the shape memory material back to the initial state (i.e. ensuring the grasping of the object). In case of using bidirectional shape memory material, it is preferable to also use a cooling system, so that the cooling down process is sufficiently fast. The open functional state may belong either to the high-temperature state of the shape memory material or to the low temperature state, the closed grasping state belonging to the other state.

In the case of shape memory materials activated by an electric signal, the shape memory material can be made to take on its predefined shape by changing the electric field (e.g. by switching it on and off). This is preferably achieved by making the shape memory material a part of the electric circuit, and electric current (or voltage) is switched into the shape memory material via the electric circuit.

Other activation signals are conceivable, such as light, magnetic field, etc.

It should be noted that not only solid shape memory materials can be used for the orthosis 20 according to the invention. Gel shape memory materials also exist (primarily polymer gels), which become cross-linked due to an activation signal, and as a result take on the pre-configured shape. For example, poly(N isopropyl-acrylamide) [PNIPAAm] based nanocomposite hydrogel cross-linked with clay mineral (Laponite XLG and XLS) is a temperature sensitive polymer with a lower critical solution temperature [LCST], i.e. its physical properties suddenly change in the vicinity of the LCST (about 34° C.). These gels display shape memory behaviour as a result of the physical cross-linking established in PNIPAAm Laponite systems and the aforementioned temperature sensitivity.

In the case of the use of shape memory polymer gels the polymer gel is preferably placed in a sealed, flexible pouch within the moving element 40, thereby making it possible for it to take on a shape conforming to the functional position of the hand 12 in cross-linked (solid) state.

In general it may be said that with the activated shape transformation of shape memory materials the moving elements may be brought into a position conforming to the functional position of the hand 12. Functional position is a concept uniformly accepted, known and used in international literature; in this position the thumb 14 and the other four fingers 15 oppose each another, but the thumb 14 is separated from the other fingers 15, therefore a space is created between the thumb 14 and the fingers 15 to accommodate the object to be grasped. In the functional position the MCP joint is bent at an approximately 45-70° and the DIP joint is bent at approximately 10-30° at the fingers 15. The wrist joint 11 may also be bent at an angle of approximately 0-30°. It should be noted that the angle ranges mentioned here are not strict values.

In the grasping position the opposing thumb 14 and fingers 15 grasp the object, and so approach each other to an extent depending on the thickness of the object, which is primarily realised through the further bending of the MCP joints at the proximal end of the fingers 15.

The material of the supporting elements 30 is preferably a flexible polyamide composite, such as the commercially available product PrimePart PLUS (PA 2221), which is suitable for use in SLS (selective laser sintering) 3D printing technology, therefore the supporting elements 30 may be simply produced using 3D printing technology, and in this way every product may be adjusted to the individual shape of the lower arm 10 and hand 12 of the given user. In the case of a preferred embodiment the orthosis 20 is made using 3D printing, which makes it significantly easier to produce custom-size orthoses 20 designed for the lower arm 10 of the user.

The orthosis 20 is preferably provided with a waterproof and electrically insulating, and preferably also heat insulating, covering, which may be, for example, a flexible ceramic coating also applied with 3D printing technology or a silicone paint coating.

The control system 22 is preferably a processor, chip, microcontroller or mini-computer, and preferably has a keyboard 24 and/or wireless communication unit 26 for inputting commands originating from an external source, as well as a display 28 for displaying information (FIG. 2). The keyboard 24 may contain just one single activation push button 25 (FIG. 7), with which the user may activate the orthosis 20 with his or her other hand, in other words create the functional position preparing for grasping. In the case of two-state shape memory material there may be a separate control push button 25 for each of the two states. Naturally, rotary switches, a touch screen or other, similar input interfaces may be conceivably instead of the push button(s) 25. The keyboard 24 and the display 28 may also be integrated in the case of a touch screen.

The wireless communication unit 26 may be any known unit that communicates with an external device, such as the user's smartphone 100, via a wireless technology standard, such as Bluetooth, zigbee or any other standard compatible with healthcare prescriptions. Software needs to be installed in the external device for communication with the orthosis 20. The remote control of various types of devices and actuators is well known to a person skilled in the art, therefore the remote control of the orthosis 20 can be implemented in an obvious way.

A remote controlled control system 22 or a control system 22 without display 28 may also be fitted to the lower side of the lower arm splint 32. In this case a place may be provided on the upper side (i.e. the wrist joint 11 side) of the lower arm splint 32 for accommodating a watch, especially a smart watch.

The orthosis 20 according to the invention further contains electric circuit elements 50 and a power source 60 for transmitting the activation signal to the shape memory material, and thereby inducing deformation of the moving elements 40. The control system 22 uses these components to control the moving elements 40.

The power source 60 preferably contains a battery 64 having a wireless charger 62. The advantage of wireless charging is that its use is convenient, furthermore, if the orthosis 20 does not contain skin sensors or stimulating electrodes, then the wireless charger makes it possible to use a waterproof and electrically insulating cover on the external surface of the orthosis 20, therefore the user may use it safely in rain, or when taking a shower, a bath or when swimming.

The electric circuit elements 50 are the usual elements used for creating circuits 51 (such as heating circuits), such as cables 52 and switches 54 used for opening and closing circuits 51, but, in addition to these, they may also include resistors, capacitors, coils, transistors, light sources, electromagnets, etc., the use of which for creating the desired circuit 51 is obvious for a person skilled in the art.

In the following an embodiment is disclosed, wherein the activation signal is a temperature signal, which is provided by the heating (or optionally cooling) of the shape memory material.

If the shape memory material is a shape memory alloy (SMA), such as NiTinol alloy, then the shape memory alloy may be connected to the power source 60 via the electric circuit elements 50, in other words the shape memory alloy itself may be a part of the circuits 51, as is shown in FIG. 7. By closing the switch 54 current starts to flow in the circuit 51, and the temperature of the shape memory alloy increases as a result of the current flowing through it.

Such an embodiment is also conceivable wherein the heat-activated shape memory material does not conduct electricity, e.g. a ceramic material, or it is not preferable to subject it to an electric field. In such a case heating filaments, as further circuit elements (not shown), connected to the power source 60 through the other electric circuit elements 50 (such as a cable 52 and a switch 54) are arranged in the moving elements 40 along the shape memory material, and the current running through these heating filaments heats the shape memory material.

The shape memory material can only return to its earlier state after the material has cooled to a temperature below the high-temperature transit temperature. The material cools passively after heating has been stopped, which may be facilitated by a (printed) flexible cooling rib system arranged around the surface. An additional possibility is if the shape memory material itself is formed as a large-surface ribbed plate, with such plates being provided in the moving elements 40.

The active orthosis 20 according to the invention is preferably supplied with an active cooling system in addition to the possibility of passive cooling, in this way the waiting time required between establishing the open functional state and the closed grasping state may be reduced. The active cooling system preferably contains Peltier cells 56 arranged in the vicinity of the shape memory materials and connected to the power source 60 by the electric circuit elements 50 (such as a cable 52 and a switch 54).

If the individual finger moving elements 46 each have a separate finger support 36, as in the case of the second embodiment illustrated in FIGS. 4 to 6, then the individual finger moving elements 46 are preferably controlled independently of each other, in other words they may be individually heated and, in the case of a cooling system being used, individually cooled. This means that the heating elements (the shape memory material itself or the heating filaments) are connected in parallel in a common circuit 51, or connected to the power source 60 via separate circuits 51. Similarly the Peltier cells 56 in the cooling system are preferably also individually controlled, and in the interest of this they are connected in parallel in a common circuit 51, or connected to the power source 60 via separate circuits 51.

Preferably, a sensor for measuring the electrical muscle activity of the lower arm extensor muscle group, preferably an electromyography (EMG) sensor 70, is arranged in the lower arm splint 32, on the upper side of the lower arm 10. The sensor 70 built into the orthosis 20 is also capable of measuring nerve (EMG) signals in the still existing and operating neural pathways, as well in any neural pathways that are not used.

The EMG sensor 70 contains at least two electrodes 71 and 72 measuring the electrical muscle activity of the lower arm extensor muscle group, and preferably a further reference electrode 73. Muscle activity can be concluded from the voltage difference between the electrodes 71 and 72. The reference electrode 73 aids the filtering out of any false signals.

In addition to this an electro-stimulation device 80, which has electro-stimulation electrodes 81 and 82, serving for the electrical muscle stimulation of the lower arm extensor muscle group is preferably arranged in the lower arm splint 32. Optionally the electro-stimulation electrodes 81 and 82 may coincide with the electrodes 71 and 72 that measure the electrical muscle activity.

The sensor 70 and the electro-stimulation device 80 also obtain their power from the power supply 60, either directly or via the control system 22, in this respect the schematic block diagram according to FIG. 7 is not decisive.

In the case of shape memory material activated with an electric signal the shape memory material itself is preferably a part of the circuit 51, and for activation an electric current (or voltage) is switched into the shape memory material using the control system 22.

In case of using a shape memory material activated with light the shape memory material may be illuminated within the moving element 40 using a light source as a circuit element 50 in order to achieve the desired shape.

In the case of the use of shape memory material activated with a magnetic field (ferromagnetic shape memory alloy) low-output electromagnets are preferably used as circuit elements 50 in order to create the magnetic field.

The use of the orthosis 20 according to the invention takes place in the following way. In the following example the operation of an orthosis 20 is activated with a temperature signal, but obviously in the case of other activation signal required the corresponding type of activation signal is produced.

The activation command for moving the orthosis 20 is received by the control system 22. The activation command may arrive via the keyboard 24, for example the user presses the activation push button 25 with her other hand. Another possibility is that the control system 22 receives the activation command via the wireless communication unit 26, from the user's smartphone 100, for example. Naturally any other device may be used for sending the activation command on which the software required for communicating with the orthosis 20 has been installed.

On the effect of the activation command the control system 22 provides the activation signal, which in the present case is the heating of the shape memory material. On the effect of the activation signal, by inducing the moving elements 40 to change their shape the shape memory materials bring the user's finger joints and wrist joints into the position corresponding to the activation signal. The position corresponding to a first activation signal may be the functional position of the finger and wrist joints, which prepares grasping.

Preferably a second activation signal serves for creating grasping, which in the case of a continuous first activation signal may also be the termination of the first activation signal. The second activation command for issuing the second activation signal may also be received through the keyboard 24 or a wireless connection. It should be noted that the first and second activation command may be inputted with the same push button 25, for example, the first push of the push button 25 serves for inducing the functional position, then the second, subsequent push is interpreted by the control system 22 as the second command activating the grasping position.

On the effect of the second activation command (including the possibility of the termination of the first activation command) the heating of the shape memory elements is terminated by the control system 22, and, optionally, active cooling is ensured, if the orthosis 20 has an active cooling system. In this latter case the cooling of the shape memory material is regulated by the control system 22 so that the shape memory materials take on the shape corresponding to the grasping position of the finger and wrist joints.

In the case of shape memory material with a unidirectional shape memory effect, by cooling the shape memory material the spastic bending muscles are permitted to involuntarily pull together the thumb 14 and the fingers 15, thereby creating grasping.

In the case of shape memory material with a bidirectional shape memory effect the thumb 14 and the fingers 14 are pulled together actively, even in absence of any muscle tone, by the cooling of the shape memory material which, as a result, takes on the bent shape corresponding to the lower transit temperature, thereby also creating grasping.

In the case of an embodiment supplied with an EMG sensor 70, the sensor 70 is used to measure the electrical muscle activity of the user's lower arm extensor muscle group, and in the case of the measurement of muscle activity aimed at tensioning the wrist and finger joints, the control system 22 interprets the signal indicating muscle activity as an activation command, and on the effect of this sends the activation signal to the shape memory material. In the present case this means that the heating of the shape memory material is controlled with the control system 22 such that by inducing a shape change in the moving elements 40 the shape memory materials bring the user's finger and wrist joints into the functional position preparing for grasping. Accordingly, in such a case the muscle activity of the user (or rather the signal of the EMG sensor 70), as activation command, induces the transformation to the functional position.

If in addition to the sensor 70 the orthosis also has an electro-stimulation device 80 installed in the lower arm splint 32 for the electrical muscle stimulation of the lower arm extensor muscle group, then in the case of measuring muscle activity aimed at tensioning the wrist and finger joints the control system 22 controls the electro-stimulation electrodes 81 and 82 so that they stimulate muscle activity in such a way as to amplify the existing personal muscle activity of the user. Preferably, the shape transformation induced by the heating/cooling of the shape memory materials is also realised in this case, because the personal muscle activity and the stimulated muscle activity both help to achieve the desired position.

In the case of using a shape memory material with a bidirectional shape memory effect the muscle activity aimed at bending the wrist and finger joints can be similarly measured and amplified with stimulated muscle activity.

The measuring of the user's muscle activity and the supplementary stimulation of muscle activity assist rehabilitation, furthermore it makes the use of the orthosis 20 easier, as it is not necessary to input an external activation command, therefore interaction with the other hand is unnecessary.

Various modifications to the above disclosed embodiments will be apparent to a person skilled in the art without departing from the scope of protection determined by the attached claims.

1. Active hand orthosis (20) for people with nerve-related paresis/paralysis of the hand, and for people with spastic paresis/paralysis of the hand, which comprises supporting elements (30) and moving elements (40) connecting the supporting elements (30), the moving elements (40) containing shape memory material, further comprising a control system (22), electric circuit elements (50) and a power source (60) for creating an activation signal inducing transformation of the shape memory material, and thereby deformation of the moving elements (40), characterised by that the moving elements (40) comprise:
a wrist joint moving element (44),
at least one finger moving element (46), and
a thumb moving element (48),
furthermore, in a state fitted to a lower arm (10) the supporting elements (30) comprise:
a lower arm splint (32) supporting a lower side of the lower arm (10), and leaving a wrist joint (11) free,
a palm support (34) connected to the lower arm splint (32) by the wrist joint moving element (44), palm support (34) being provided at the proximal end of the fingers (15) opposable to the thumb (14),
at least one finger support (36) connected to the palm support (34) by the at least one finger moving element (46), at least one finger support (36) being provided at the middle bone of the fingers (15) opposable to the thumb (14), and
a thumb support (38) connected to the lower arm splint (32) by the thumb moving element (48), the thumb support (38) extending from the proximal end of the thumb (14) on the palm (13) to the lower bone of the thumb (14).

2. Active orthosis (20) according to claim 1, characterised by that the lower arm splint (32) has a fold-out strap part (32a) that encompasses the lower arm (10) when fitted to the lower arm (10), and a locking system, preferably a magnetic lock, is provided on the strap part (32a) for securing the lower arm splint (32) in its position embracing the lower arm (10).

3. Active orthosis (20) according to claim 1 or 2, characterised by comprising a common finger support (36) provided as a single element for the fingers (15) opposable to the thumb (14).

4. Active orthosis (20) according to claim 1 or 2, characterised by that it contains one finger support (36) for each of the fingers (15) opposable to the thumb (15), each of which is connected to the first palm support (34) by finger moving elements (46) that are controllable independently of each other.

5. Active orthosis (20) according to any of claims 1 to 4, characterised by that the control system (22) contains an element selected from a group consisting of a processor, a chip, a microcontroller and a mini-computer.

6. Active orthosis (20) according to any of claims 1 to 5, characterised by that the control system (22) has a keyboard (24) and/or communication unit (26) for inputting external commands.

7. Active orthosis (20) according to any of claims 1 to 6, characterised by that the material of the supporting elements (30) is flexible polyamide composite.

8. Active orthosis (20) according to any of claims 1 to 7, characterised by that it is provided with a waterproof, heat-insulating and electrically insulating covering, preferably a flexible ceramic coating or silicone paint coating.

9. Active orthosis (20) according to any of claims 1 to 8, characterised by that the supporting elements (30) are produced using 3D printing technology.

10. Active orthosis (20) according to any of claims 1 to 9, characterised by that a sensor (70) is arranged in the lower arm splint (32) for measuring the electrical muscle activity in the lower arm extensor muscle group, preferably an electromyography (EMG) sensor.

11. Active orthosis (20) according to claim 10, characterised by that electro-stimulation electrodes (81, 82) are arranged in the lower arm splint (32) for electrical muscle stimulation of the lower arm extensor muscle group.

12. Active orthosis (20) according to any of claims 1 to 11, characterised by that the activation signal is a temperature signal and heating filaments connected to the power source (60) via the electric circuit elements (50) are arranged in the orthosis (20) for heating the shape memory materials.

13. Active orthosis (20) according to claim 12, characterised by that it is provided with a cooling system serving for cooling the shape memory material, which preferably contains Peltier cells (56) arranged next to the shape memory materials, and connected to the power source (60) via the electric circuit elements (50).

14. Active orthosis (20) according to any of claims 1 to 11 characterised by that the activation signal is an electric signal, and the shape memory material is connected to the power source (60) via the electric circuit elements (50).

15. Active orthosis (20) according to claim 1 characterised by that the shape memory material is selected from a group consisting of shape memory metals, shape memory ceramics, shape memory polymers and shape memory polymer gels.

16. Method for the use of the active hand orthosis (20) according to claim 1, characterised by receiving an activation command by the control system (22), creating an activation signal by the power source (60) and the electric circuit elements (50) on the effect of the activation command that induces the transformation of the shape memory materials, and thereby bringing the user's finger and wrist joints (11) into a position according to the activation signal through the deformation of the moving elements (40).

17. Method according to claim 16, characterised by that the position according to the activation signal is the functional position of the finger and wrist joints (11).

18. Method according to claim 17, characterised by that is a temperature signal, and providing the activation signal by heating and/or cooling the shape memory material.

19. Method according to any of claims 16 to 18, characterised by providing a sensor (70), preferably an electromyography (EMG) sensor, in the lower arm splint (32) for measuring the electrical muscle activity in the lower arm extensor muscle group, measuring the electrical activity of the user's lower arm extensor muscle group therewith, and receiving by the control system (22) the sensor's (70) signal of muscle activity for tensioning the wrist and finger joints (11) as the activation command.

20. Method according to claim 19, characterised by providing electro-stimulation electrodes (81, 82) in the lower arm splint (32) for electrical muscle stimulation of the lower arm extensor muscle group, and controlling the electro-stimulation electrodes (81, 82) by the control system (22) in case of receiving the activation command such that a stimulated muscle activity amplifies the personal muscle activity.
